(19) United States
(12) Patent Application Publication
(10) Pub. No.: US 2023/0087044 A1
(43) Pub. Date: Mar. 23, 2023

(54) SYSTEMS, METHODS, AND APPARATUSES FOR CLEANING A CAMERA DURING A MEDICAL PROCEDURE

(71) Applicant: TITAN MEDICAL INC., Toronto (CA)

(72) Inventors: Perry A. Genova, Chapel Hill, NC (US); Dustin Christopher Simons, Chapel Hill, NC (US)

(21) Appl. No.: 17/933,982

(22) Filed: Sep. 21, 2022

Related U.S. Application Data
(60) Provisional application No. 63/247,186, filed on Sep. 22, 2021.

(51) Int. Cl. A61B 90/70; A61B 90/00; A61B 90/30; A61B 34/35; A61B 34/37; A61B 34/20; H04N 5/225

(52) U.S. Cl. CPC A61B 90/70; A61B 90/36; A61B 90/30; A61B 90/39; A61B 34/35; A61B 34/37; A61B 34/20; H04N 5/2252; A61B 2034/2057; A61B 2090/363

(57) ABSTRACT

One or more camera lenses used for robotic surgery can be cleaned without the need to remove and reinsert one or more cameras. The approaches described herein facilitate cleaning the one or more lenses or one or more protective windows on demand and without the need to remove and reinsert the one or more cameras. Cleaning can include one or more of washing or wiping the one or more lenses or one or more protective windows.

### CROSS-REFERENCE TO RELATED APPLICATIONS

This application claims priority to U.S. Provisional Patent Application No. 63/247,186, filed on Sep. 22, 2021, which is incorporated by reference in its entirety.

### TECHNICAL FIELD

This disclosure relates generally to positioning a camera for imaging and more particularly to positioning a camera inside a body cavity of a patient for capturing images during a medical procedure and cleaning the camera.

### DESCRIPTION OF RELATED ART

Miniaturized cameras are used during investigative medical procedures and surgical procedures, such as laparoscopic surgery and computer assisted robotic surgery, to produce images of a site of the procedure within a body cavity of the patient. A camera generally includes an illumination source for illuminating the site of the procedure and a lens for capturing images of the site. Known camera systems suffer from a variety of shortcomings, including large size, poor resolution, obstacles with being sterilized, lack of reliability, difficulties with being cleaned or replaced during medical procedure, and the like. The present disclosure overcomes these and other problems associated with known camera systems, methods, and apparatuses.

### SUMMARY

A visualization device for a robotic surgery apparatus can include a housing configured to be removably attached to a mounting interface of the robotic surgery apparatus. The housing can include first and second openings positioned on an exterior of the housing. The visualization device can include a camera shaft including a distal end comprising at least one camera that includes at least one lens covered by at least one window. The distal end can be configured to be inserted through the first opening in the housing, pass through an interior of the housing, and exit the housing through the second opening in the housing. The distal end can be configured to extend away from the housing toward a region of interest outside the housing and retract back toward the housing. The visualization device can include a chamber positioned in the interior of the housing and configured to receive at least a portion of the distal end. The chamber can be configured heat up and clean the at least one window of the at least one camera.

The visualization device of the preceding paragraph and/or any of the visualization devices disclosed herein can include one or more of the following features. The visualization device can include an electronic circuitry positioned in the interior of the housing and proximal to the chamber. The electronic circuitry can be configured generate heat that regulates temperature of the chamber to at least one of prevent fogging of the at least one lens or the at least one window or clean the at least one window of the at least one camera. The electronic circuitry can include an illumination source configured to produce illumination for the region of interest. The chamber can be positioned in a conduit comprising the first and second openings. The visualization device can include first and second seals configured to seal off a portion of the conduit to form the chamber. The chamber can be configured to retain a fluid for cleaning the at least one window of the at least one camera. The housing can include first and second openings positioned at least partially on the exterior of the housing. The first opening can be configured to permit introduction of the fluid into the chamber. The second opening can be configured to permit removal of the fluid from the chamber.

The visualization device of any of the preceding paragraphs and/or any of the visualization devices disclosed herein can include one or more of the following features. The camera shaft can include a proximal end attached to the housing. The camera shaft can include a fiducial marker. The visualization device can include a sensor configured to detect the fiducial marker. The visualization device can include an electronic processing circuitry. The electronic processing circuitry can be configured to cause the camera shaft to be retracted responsive to receiving a user input, wherein the distal end is positioned at a first position prior to retracting the flexible camera shaft. The electronic processing circuitry can be configured to cause stopping of the retraction of the camera shaft responsive to receiving a signal from the sensor indicating detection of the fiducial maker by the sensor, wherein causing the retraction of the camera shaft to be stopped causes the distal end to be positioned at a second position within the chamber. The electronic processing circuitry can be configured to cause extension of the camera shaft to return the distal end to the first position.

The visualization device of any of the preceding paragraphs and/or any of the visualization devices disclosed herein can include one or more of the following features. The visualization device can include a position sensor configured to monitor position of the distal end. The visualization device can include an electronic processing circuitry. The electronic processing circuitry can be configured to cause the camera shaft to be retracted responsive to receiving a user input and further responsive to an output of the position sensor, wherein the distal end is positioned at a first position prior to retracting the camera shaft. The electronic processing circuitry can be configured to cause stopping of the retraction of the camera shaft responsive to the output of the position sensor, wherein causing the retraction of the camera shaft to be stopped causes the distal end to be positioned at a second position within the chamber. The electronic processing circuitry can be configured to, responsive to the output of the position sensor, cause the camera shaft to be extended to position the distal end at the first position.

The visualization device of any of the preceding paragraphs and/or any of the visualization devices disclosed herein can include one or more of the following features. The housing can be configured to receive a cleaning strip that includes at least one cleaning portion configured to wipe the at least one window. The cleaning strip can include alternating wiping portions and lumen portions. A wiping portion can be configured to wipe the at least one window and a lumen portion configured to allow the camera shaft to pass through and enter the first opening. Disclosed is a kit including the visualization device of any of the preceding paragraphs and/or any of the visualization devices disclosed herein and the cleaning strip. The cleaning strip can be arranged on a reel configured to be positioned above or below the device.

An insertion device for a robotic surgery apparatus can include a housing. The housing can include a first portion including a camera channel positioned in an interior of the first portion and extending along at least a portion of the first portion. The camera channel can be configured to permit insertion and removal of a primary camera shaft. The primary camera shaft can include a primary camera with at least one lens covered by at least one window. The housing can include a second portion including a passage configured to permit the primary camera shaft to pass through. The passage can be aligned with the camera channel to permit at least a portion the primary camera shaft to enter the camera channel, pass through the camera channel, and exit the camera channel. The passage can include an opening positioned on an exterior of the housing. The housing can include a chamber positioned in the passage and configured to heat up and clean the at least one window of the primary camera.

The insertion device of the preceding paragraph and/or any of the insertion devices disclosed herein can include one or more of the following features. The insertion device can include an electronic circuitry positioned in the interior of the second portion and proximal to the chamber. The electronic circuitry can be configured generate heat that regulates temperature of the chamber to at least one of: prevent fogging of the at least one lens or the at least one window or clean the at least one window of the at least one camera. The first portion can include a second camera channel configured to receive a secondary camera. The electronic circuitry can include an illumination source configured to produce illumination for one or more of the primary camera or the secondary camera. The secondary camera can be non-removable. The insertion device can include first and second seals configured to seal off a portion of the passage to form the chamber. The chamber can be configured to retain a fluid for cleaning the at least one window of the at least one camera. The housing can include first and second openings positioned at least partially on the exterior of the first portion. The first opening can be configured to permit introduction of the fluid into the chamber. The second opening can be configured to permit removal of the fluid from the chamber.

The insertion device of any of the preceding paragraphs and/or any of the insertion devices disclosed herein can include one or more of the following features. The primary camera can include a fiducial marker. The insertion device can include a sensor configured to detect the fiducial marker. The insertion device can include an electronic processing circuitry. The electronic processing circuitry can be configured to cause the primary camera shaft to be retracted responsive to receiving a user input, wherein the distal end is positioned at a first position prior to retracting the primary camera shaft. The electronic processing circuitry can be configured to cause stopping of the retraction of the primary camera shaft responsive to receiving a signal from the sensor indicating detection of the fiducial maker by the sensor, wherein causing the retraction of the primary camera shaft to be stopped causes the distal end to be positioned at a second position within the chamber. The electronic processing circuitry can be configured to cause extension of the primary camera shaft to return the distal end to the first position.

The insertion device of any of the preceding paragraphs and/or any of the insertion devices disclosed herein can include one or more of the following features. The insertion device can include a position sensor configured to monitor position of the distal end. The insertion device can include an electronic processing circuitry. The electronic processing circuitry can be configured to cause the primary camera shaft to be retracted responsive to receiving a user input and further responsive to an output of the position sensor, wherein the distal end is positioned at a first position prior to retracting the primary camera shaft. The electronic processing circuitry can be configured to cause stopping of the retraction of the primary camera shaft responsive to the output of the position sensor, wherein causing the retraction of the camera shaft to be stopped causes the distal end to be positioned at a second position within the chamber. The electronic processing circuitry can be configured to, responsive to the output of the position sensor, cause the camera shaft to be extended to position the distal end at the first position.

The insertion device of any of the preceding paragraphs and/or any of the insertion devices disclosed herein can include one or more of the following features. The housing can be configured to receive a cleaning strip that includes at least one cleaning portion configured to wipe the at least one window. The cleaning strip can include alternating wiping portions and lumen portions. A wiping portion can be configured to wipe the at least one window and a lumen portion configured to allow the primary camera shaft to pass through and enter the passage through the opening. Disclosed is a kit including the insertion device of any of the preceding paragraphs and/or any of the insertion devices disclosed herein and the cleaning strip. The cleaning strip can be arranged on a reel configured to be positioned above or below the device.

Any of the insertion devices of any of preceding paragraphs and/or described below can be used with any of visualization devices and/or robotic surgery systems described herein.

In some cases, a robotic surgery apparatus as described and/or illustrated is provided. In some cases, a visualization device as described and/or illustrated is provided. In some cases, an insertion device as described and/or illustrated is provided.

In some cases, a method of using and/or operating a robotic surgery apparatus or any of its components as described and/or illustrated is provided. In some cases, a method of using and/or operating a visualization device as described and/or illustrated is provided. In some cases, a method of using and/or operating an insertion device as described and/or illustrated is provided.

Any of the methods of any of preceding paragraphs and/or described below can be used with any of insertion devices, visualization devices, and/or robotic surgery systems and/or any of the methods of operating and/or using such devices and/or systems described herein.

### BRIEF DESCRIPTION OF THE DRAWINGS

Embodiments of the present disclosure will now be described hereinafter, by way of example only, with reference to the accompanying drawings in which:

FIG. 1 illustrates a robotic surgery system in accordance with some embodiments;

FIG. 2 illustrates an insertion and visualization device according to some embodiments;

FIGS. 3A-3B illustrate an insertion device according to some embodiments;

FIGS. 4A-4C illustrate a visualization device according to some embodiments;

FIGS. 5A-5C and 6 illustrate camera lens cleaning arrangements according to some embodiments.

### DETAILED DESCRIPTION

### Overview

When performing medical procedures (for example, with assistance of surgery using a robotic surgical system) one or more instruments can be inserted into a body cavity of a patient. The insertion process has some risk since instruments may inadvertently damage organs or tissue while being inserted. Incorrect positioning of the one or more instruments in the body cavity may also result in a limited range of motion within the body cavity.

As an example, when performing abdominal surgery, at least one incision would be made in a body wall of the patient's abdomen. A trocar or other access port, may then be inserted through the incision. A camera can be first inserted through the access port and used by a surgeon to capture and relay stereoscopic images of a surgical site. One or more instruments can be inserted following the camera insertion. Views provided by the camera facilitate insertion of the one or more instruments and their manipulation of the surgical site.

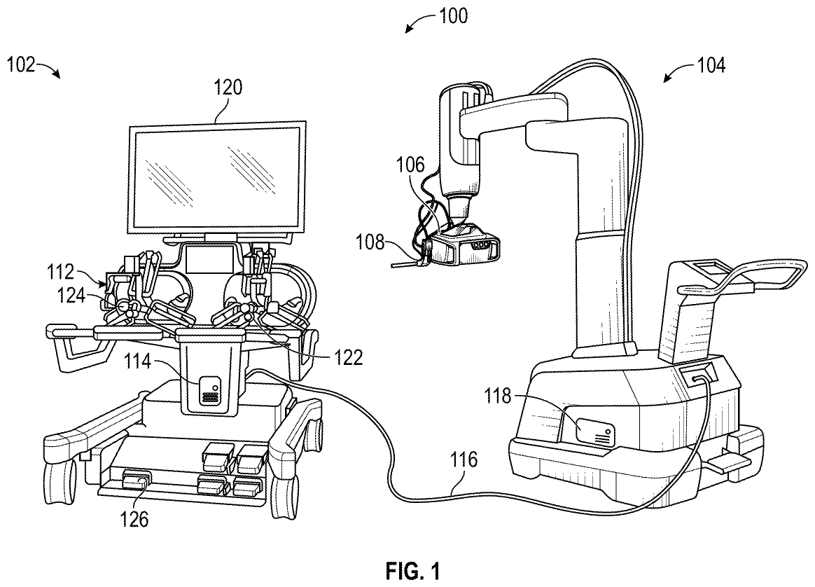

Referring to FIG. 1, a robotic surgery system is shown generally at 100. In some implementations, the robotic surgery system 100 can be configured to facilitate a medical procedure performed via a single incision. A single access port can be inserted into the incision to provide access for one or more instruments and cameras. In some cases, the robotic surgery system 100 can be configured to facilitate a medical procedure performed via multiple incisions and/or multiple access ports.

The system 100 can include a workstation 102 and a patient cart 104. The patient cart 104 can include a central unit or drive unit 106 to which instrument insertion and visualization devices 108 can be attached or mounted. The workstation 102 can include an input device 112 that receives operator input and produces input signals and may also be configured to generate feedback to the operator. The feedback can be visual, auditory, haptic, or the like. The input device 112 can be implemented using a haptic interface available from Force Dimension, of Switzerland, for example.

The workstation 102 can further include electronic circuitry 114 in communication with the input device 112 for receiving the input signals and generating control signals for controlling the robotic surgery system, which can be transmitted to the patient cart 104 via an interface cable 116. In some cases, transmission can be wireless and interface cable 116 may not be present. The electronic circuitry 114 can include one or more processors or controllers. The electronic circuitry 114 can function as a master for controlling movement of one or more surgical instruments or cameras mounted to the patient cart 104. The patient cart can include electronic circuitry 118, which can include one or more processors or controllers. The electronic circuitry 118 can function as a slave and be controlled by the electronic circuitry 114. Communication between the electronic circuitry 114 of the workstation 102 and the electronic circuitry 118 of the patient cart 104 may wired (such as, via the cable 116) or wireless. The workstation 102 may be located remotely from the patient cart 104, such as outside the operating room or in a non-sterile area of the operating room.

The input device 112 can include right and left hand controllers 122 and 124, which are configured to be grasped by the operator's hands and moved to produce input signals at the input device 112. The patient cart 104 can include electronic circuitry 118, which can include one or more processors or controllers. The electronic circuitry 118 can receive control signals from the electronic circuitry 114 and produces slave control signals operable to control the instrument insertion and visualization devices 108 and one or more instruments (and their respective end effectors) during a surgical procedure. The one or more instruments can include dexterous tools, such as grippers, needle drivers, staplers, dissectors, cutters, hooks, graspers, scissors, coagulators, irrigators, suction devices, which are used for performing a surgical procedure. While both master and slave processor circuits are illustrated, in other embodiments a single processor circuit may be used to perform both master and slave functions. The workstation 102 can also include a user interface, such as a display 120 (which can be referred to as a primary display) in communication with the electronic circuitry 114 for displaying information (such as, body cavity images) for a region or site of interest (for example, a surgical site, a body cavity, or the like) and other information to an operator. The workstation 102 can include an auxiliary display 123 (which can be referred to as a secondary display) for displaying auxiliary surgical information, for example, patient medical charts, pre-operation images, images acquired during operation, or the like. In some cases, the secondary display 123 may be a touch display and may also be configured to display graphics representing additional inputs for controlling the workstation 102 or the patient cart 104. The workstation 102 can also include one or more controllers, such as one or more pedals 126, for controlling the robotic surgery system. For example, one or more pedals 126 can include a clutch pedal that allows repositioning one or more controllers 122 or 124 without corresponding movement of the associated instrument.

The workstation 102 can include electronic circuitry (of which the electronic circuitry 114 can be part of), configured to, among other things, control one or more of the display 120 or the secondary display 123. The electronic circuitry can receive image data from one or more cameras described herein and operate one or more of the display 120 or the secondary display 123 to display the image data. The electronic circuitry can process the image data, such as filter, decode, encode, recode, compress, decompress, combine, or the like.

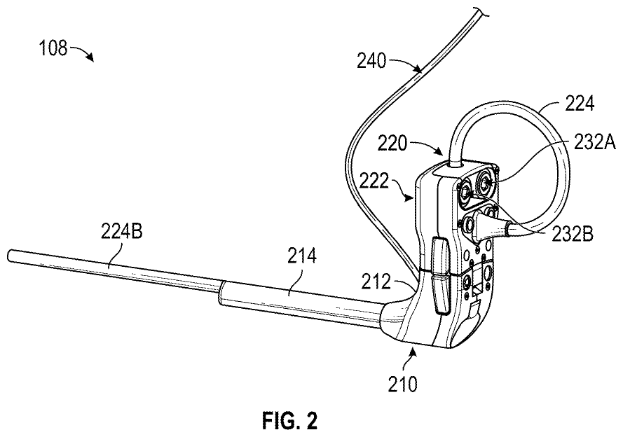

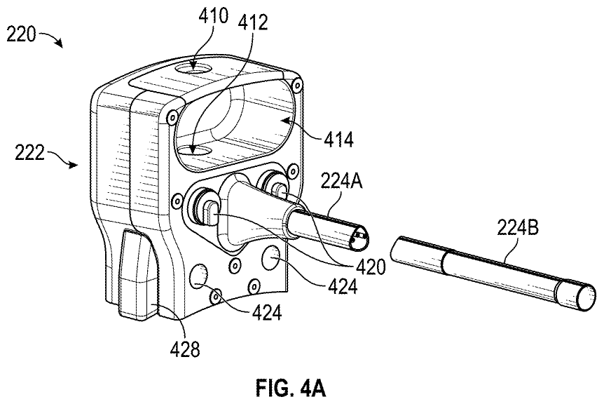

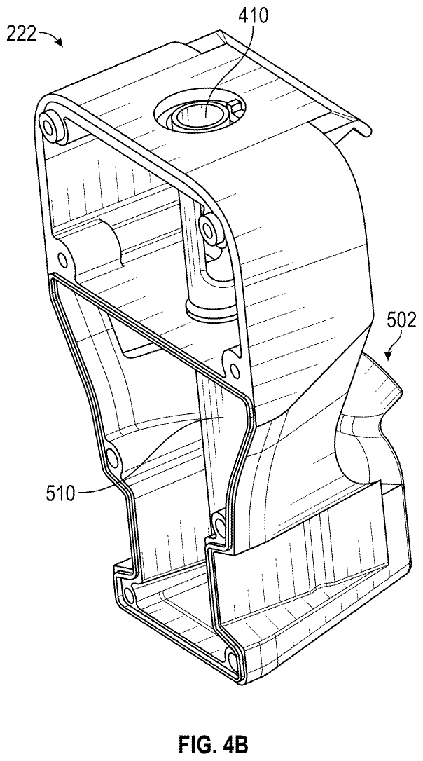

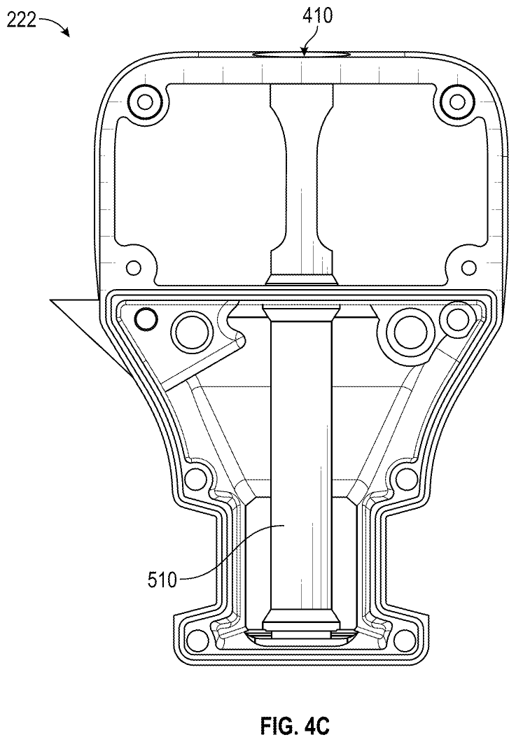

Referring to FIG. 2, insertion and visualization devices 108 can include an insertion device 210 and a visualization device 220. The insertion device 210 can include a housing 212 and a plurality of passages, lumens, tubes, conduits, or channels 214 for inserting and guiding one or more instruments. The plurality of channels 214 can be enclosed in another housing. The two housings can be connected. As is illustrated, the plurality of channels, such as radial channels, can be formed within a housing, which can be radially shaped. The plurality of channels 214 can also permit insertion of a camera lumen, cable, elongate shaft, or tube 224. As is illustrated, a distal end 224B of the camera tube can extend beyond the housing including the plurality of channels 214. At least a portion of the distal end 224B can be positioned near or in the site of interest. One or more cameras can be positioned at the distal end 224B. The camera tube 224 can also include a proximal end (illustrated as 224A in FIG. 4A) and a distal end (illustrated as 224B in FIG. 2). In some implementations, a channel of the plurality of channels 214 can house or support a camera in addition to or instead of the one or more cameras of the camera tube 224.

The visualization device can include a housing 222 to which the proximal end 224A of the camera tube can be removably (or non-removably) attached. The housing 222 can include an opening in which a one or more drivers, such as at least one of 232A or 232B, can be positioned. The one or more drivers can move the camera tube 224 through the opening in the housing 222 and a channel of the plurality of channels 214 so that the distal end 224B extends away from one or more of the housings 212 or 222 or retracts back toward or into one or more of the housings 212 or 222. The camera tube 224 can form a loop around at least a portion of the housing 222 as illustrated in FIG. 2. The diameter of the loop can be increased when the distal end 224B is retracted toward or into one or more of the housings 212 or 222 and be decreased when the distal end 224B is extended away from one or more of the housings 212 or 222.

One or more cables 240 can be used to transmit control signals and data, such as analog or digital image data provided by the one or more cameras positioned at the distal end 224B or in the insertion device 210, to the patient cart 104. Control signals and data can be communicated to and from the electronic circuitry (for example, via the electronic circuitry 118 as described herein). One or more cables 240 can transmit power to the one or more cameras. One or more cables 240 can be plugged into a port positioned on the patient cart 104. In some cases, transmission can be wireless and one or more cables 240 may not be present.

The camera tube 224 can be rigid or flexible (such as, bendable). At least a portion of the camera tube 224 can be flexible or substantially flexible in order to form a loop and/or be guided through the one or more openings and/or channels are described herein. In some cases, looping the camera tube 224 upward around at least the portion of the housing 222 as described can permit the camera tube to have sufficient length for reaching near and/or into the site of interest, while eliminating or reducing the risk of the camera tube 224 coming into contact with non-sterile object, such as the floor.

### Insertion Device

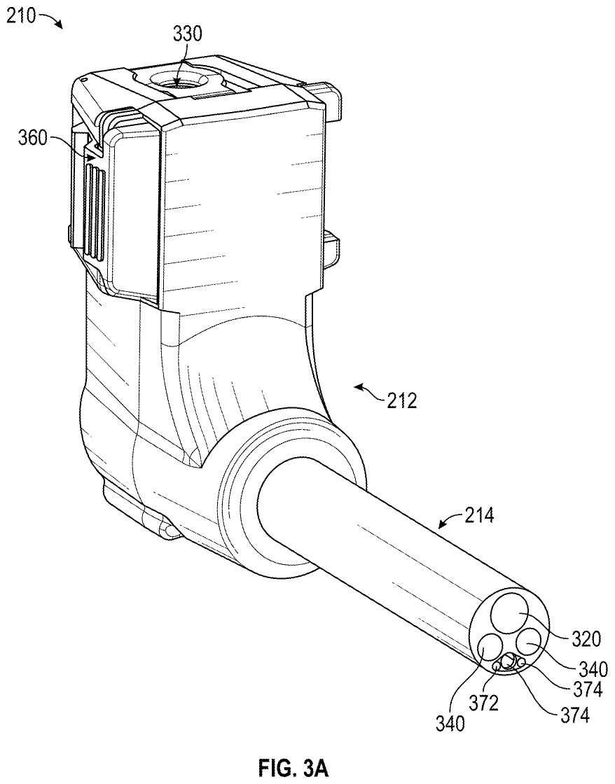

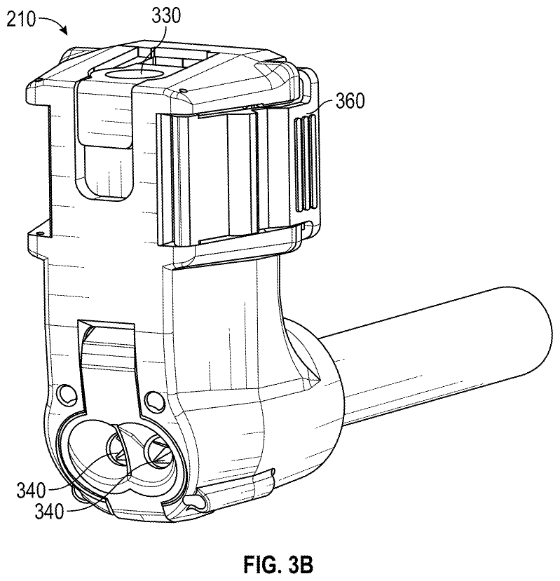

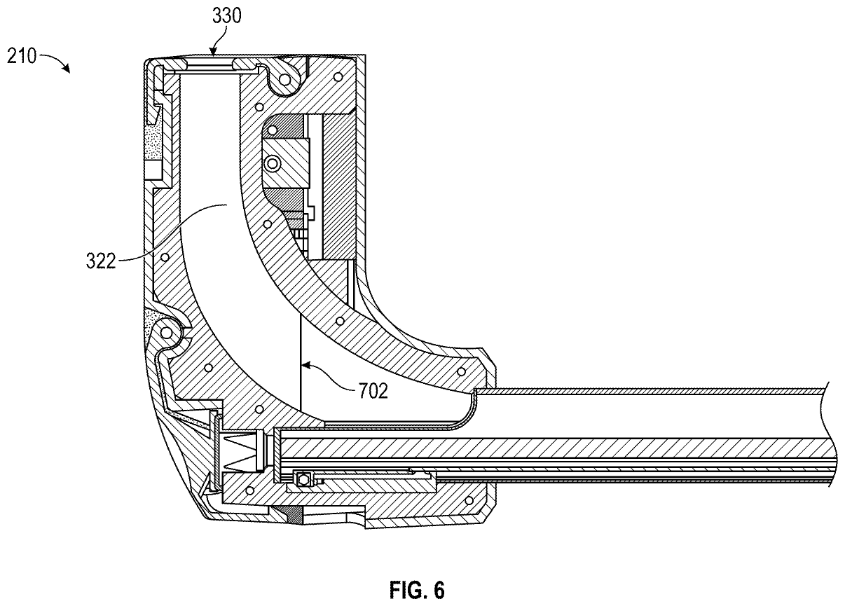

FIG. 3A illustrates a front perspective view of the insertion device 210. The housing 212 of the insertion device can include an opening 330 configured (for example, sized and/or shaped) to permit the camera tube 224 to pass through the housing 212. The opening 330 can include a seal, which may be covered by a closure (such as a latch), to prevent ingress of fluid, gas, or solids into the insertion device 210 and/or prevent backflow of fluid, gas, or solids from the insertion device. Any of the seals described herein can include one or more valves, such as a duckbill valve. As illustrated in FIG. 6 showing a cross-section view of the insertion device 210, the housing 212 can include an interior passage 322 connecting the opening 330 to a channel 320 configured (for example, sized and/or shaped) to permit the camera tube 224 to pass through the channel. The interior passage 322 can be a channel positioned in an interior of the housing. The interior passage 322 can be bent or curved to facilitate various positional configurations of the visualization device 220 with respect to the insertion device 210 and in particular the housing 222 with respect to housing 212. The interior passage 322 can include an opening that aligns with or includes the opening 330 and another opening that aligns with or includes opening of the channel 320. In some cases, sealing material can be used on or around the interior passage 322 in addition to or instead of the seal in the opening 330. As illustrated in FIG. 2, the distal end 224B of the camera tube 224 can exit the channel 320 and extend away from the insertion device 210 toward a site of interest, such as a surgical site, body cavity, wound, or the like. Also, the distal end 224B of the camera tube 224 can retract toward or into the channel 320 toward the insertion device 210 and away from the site of interest.

The plurality of channels 214 can include one or more instrument channels 340 configured (for example, sized and/or shaped) to permit one or more instruments to pass through and extend away from the insertion device 210 toward the site of interest. As is illustrated, there can be two channels for left and right instruments.

In some cases, the interior passage 322 includes at least a portion with a central axis parallel to a central axis of the one or more instrument channels 340. The interior passage 322 can include at least a portion (for example, the curved portion illustrated in FIG. 6) with a central axis not parallel to a central axis of the one or more instrument channels 340.

The plurality of channels 214 can include a channel 310 for one or more cameras of the insertion device 210. In some implementations, a camera can be positioned at a distal end of the insertion device 210. Such one or more cameras (which can be referred to as a secondary camera) can facilitate positioning adjacent to or insertion into the site of interest of at least one of one or more instruments or at least one of the one or more cameras of the visualization device 220 (such cameras can be referred to as a primary camera, which can be endoscope or endoscopic cameras). The secondary camera can include a substantially flexible or substantially rigid lumen, cable, or elongate shaft that is inserted into the channel 310. The secondary camera can be integrated with the insertion device 210 or be removable. An opening of the channel 310 can include one or more seals, which may be covered by a closure (such as a latch), to prevent ingress of fluid, gas, or solids. In some cases, sealing material can be used on or around the opening of the channel 310 in addition to or instead of the seal(s) in the opening.

One or more illumination sources can be provided, for instance, at the distal end of the insertion device 210. For instance, an illumination source 372 can be positioned as shown. The illumination source 372 can include one or more fibers (which can have a high numerical aperture). The illumination source 372 can provide white (or other visible or invisible) light. As another example, an illumination source 374 can be positioned as shown. The illumination source 374 can provide indocyanine green (ICG) illumination. In use, the illumination sources 372 and 374 can be switched as needed.

In some cases, the primary camera can be a stereo or stereoscopic camera, which can produce three-dimensional representation of at least a portion of the site of interest, and the secondary camera can be a two-dimensional camera. The secondary camera can have lower resolution than the primary camera. For example, the secondary camera can have 1920×1080 pixels (or 1080p) resolution. The primary camera can have resolution of 1080p, 4K, 8K, or the like. The channel 310 for the secondary camera can be smaller in size (such as, narrower or having smaller diameter) than the channel 320 for the primary camera. The secondary camera may also include an illumination source or device for illuminating the site of interest. The illumination device can be incorporated as part of the secondary camera such that the illumination device and a lens system of the secondary camera all fit within the diameter of the channel 310. In some cases, the illumination device can include optical fiber(s). For example, the illumination device can be an annular system with strands of fiber wrapping around a lens system so that illumination is provided to the site of interest, for instance, using known means of fiber illumination.

In some cases, close proximity of the instrument channels 340 to one or more camera channels 310 or 320 can facilitate single port surgery (although, multiple port surgery is also contemplated).

The housing 212 can include one or more attachment mechanisms 360. For example, the one or more attachment mechanisms 360 can be buttons positioned on opposite sides of the housing 212. Although a single button is illustrated in FIGS. 3A and 3B, two buttons positioned on the opposite sides of the housing 212 can be utilized. The buttons can be configured to removably attach the insertion device 210 to a mounting interface of the drive unit 106 (or, in some cases, additionally or alternatively to the housing 222 of the visualization device 220). Pushing the buttons can release the insertion device 210 from the mounting interface (and/or the housing 222 of the visualization device 220). The one or more attachment mechanisms 360 can permit attachment to and release of the insertion device 210 from supporting pins of the mounting interface (and/or the housing 222).

FIG. 3B illustrates a rear perspective view of the insertion device 210. Openings of the one or more instrument channels 340 can include one or more seals, which may be covered by a closure (such as a latch), to prevent ingress of fluid, gas, or solids. In some cases, sealing material can be used on or around at least one of the one or more openings of the one or more instrument channels 340 in addition to or instead of the seal(s) in the one or more openings. The housing 212 can include one or more openings 350 for receiving one or more supporting rods of pins, which can be positioned on the mounting interface. The one or more attachment mechanisms 360 can permit attachment to and release of the insertion device 210 from the supporting pins (and/or from the visualization device 220). For example, the one or more attachment mechanisms 360 can activate or release a latch or lock, such as a cam lock, cam lock with a spring, or the like.

### Visualization Device

FIG. 4A illustrates a front perspective view of the visualization device 220. The housing 222 of the visualization device can include openings 410 and 412 configured (for example, sized and/or shaped) to permit the camera tube 224 to pass through. As illustrated, the proximal end 224A of the camera tube 224 (illustrated for convenience without a middle portion) can be attached to the housing. The camera tube 224 can loop around at least the portion of the housing 222 when the distal end 224B is inserted through one or more of the openings 410 and 412 (see FIG. 2). The openings 410 and 412 can be aligned to permit the camera tube 224 to pass through. A bottom opening (illustrated as 502 in FIG. 4B) aligned with the opening 412 can be positioned on the bottom of the housing 222 to permit the camera tube 224 to exit the housing 222 after passing through an interior portion of the housing (such as, the interior portion illustrated in FIGS. 4B and 4C). This bottom opening 502 can be positioned adjacent to (such as over or on top of) the opening 330 in the housing of the insertion device 210 when the visualization device 220 is positioned adjacent to and/or attached to the insertion device. One or more of the openings 410, 412, or the bottom opening can include a seal, which may be covered by a closure (such as a latch) as described herein.

With reference to FIGS. 4B and 4C, which illustrate a rear cross-sectional view of the visualization device 220, a conduit 510 can connect the openings 410 and 412 to the opening 502. The conduit 510 can be configured (for example, sized and/or shaped) to enclose the camera tube 224 and permit at least the distal end 224B of the camera tube to pass through. For reasons described herein, the conduit 510 can be made of heat-conducting material.

The housing 222 can include a drive opening 414. The drive opening can be positioned on a side of the housing 222 (for example, the back of the housing) that attaches to the mounting interface of the drive unit 106 as described herein. The drive opening 414 can be configured (for example, sized and/or shaped) to receive one or more drivers (at least one of 232A or 232B), such as a plurality of drive rollers as described herein (see, for example, FIG. 2). With reference to FIG. 2, the plurality of drive rollers can include right drive roller 232A and left drive roller 232B (collectively, referred to as 232). When inserted through the opening 410, the camera tube 224 is positioned between the right and left drive rollers 232A and 232B and contacts the drive rollers. The drive rollers 232 can contact, grip, or abut the camera tube 224. The drive rollers can advance the camera tube 224 down or retract it up through the drive opening 414. Movement of the drive rollers 232 in a first direction can advance the camera tube 224 forward or down through the drive opening 414 in order to advance the distal end 224B toward the site of interest. For example, the right driver roller 232A can spin counterclockwise and the left drive roller 232B can spin clockwise in order to advance the camera tube 224 forward. Such combination of the counterclockwise and clockwise movement of the drive rollers can constitute the first direction. Movement of the drive rollers 232 in a second direction can retract the camera tube 224 backward or up through the drive opening 414 in order to retract the distal end 224B away from the site of interest. For example, the right drive roller 232A can spin clockwise and the left drive roller 232B can spin counterclockwise in order to retract the camera tube 224 backward. Such combination of the clockwise and counterclockwise movement of the drive rollers can constitute the first direction. For each of the right and left drive rollers, movement in the second direction can be opposite to movement in the first direction even in cases where drive rollers spin in opposite directions during movement in the first and/or section direction.

Drive rollers 232 can have an external surface that is made out of and/or is covered by soft material, such as rubber, foam, or the like, that grips an external surface of the camera tube 224 in order to one or more of advance or retract the camera tube. In some implementations, a portion of the camera tube 224 positioned between the drive rollers 232 can slip along the drive rollers, and as a result the camera tube would not be advanced or retracted. For example, slipping can be advantageous when a user's limb becomes caught in the loop formed by the camera tube 224 or in case of malfunction to prevent or lessen the risk of injury to the user or damage to one or more of the camera tube 224, the visualization device 220, the insertion device 210, or any other part of the system 100. At least one of one or more of the material on the external surface of the drive rollers 232 or on an external surface of the camera tube 224 or a surface pattern on the surface of one or more of the external surface of the drive rollers 232 or the external surface of the camera tube 224 can be selected to have a friction coefficient that results in slippage in case force on the camera tube exceeds a maximum force, such as, a maximum frictional force. The maximum frictional force can depend on one or more of the friction coefficient between the drive rollers 232 and camera tube 224 or a clamping force between the drive rollers 232 and camera tube 224. In some cases, the maximum frictional force can be 5N or less or more, 7N or less or more, 10N or less or more, or the like. Surface pattern on the external surface of the drive rollers 232 (and/or the external surface of the camera tube 224) can affect the friction coefficient. For example, ribbed surface pattern, toothed surface pattern, or the like can increase the friction coefficient compared to a smooth or substantially smooth surface pattern.

At least a portion of the distal end 224B of the camera tube 224 can articulate to permit viewing of at least a portion of the site of interest. The housing 222 can include one or more actuators 420 configured to control movement of the distal end 224B of the camera tube 224, which can include one or more cameras. In some cases, a first actuator can control pitch or tilt (up/down movement) of the distal end 224B, and a second actuator can control yaw or pan (left/right movement) of the distal end 224B. The first and second actuators can control movement of the distal end 224B by manipulating links positioned in the interior of the camera tube 224.

The housing 222 can include one or more attachment mechanisms 428. For example, the one or more attachment mechanisms 428 can be buttons positioned on opposite sides of the housing 222. The buttons can be configured to removably attach the visualization device 220 to the mounting interface of the drive unit 106 (or, in some cases, additionally or alternatively to the housing 212 of the insertion device 210). Pushing the buttons can release the insertion device 210 from the mounting interface (and/or the housing 212). The one or more attachment mechanisms 428 can permit attachment to and release of the visualization device 220 from one or more supporting rods or pins (and/or the housing 212). As described herein, the one or more attachment mechanisms 428 can activate or release a lock, such as a cam lock, cam lock with spring, or the like. The housing 222 can include one or more openings 424 for receiving one or more the supporting pins that can be positioned on the mounting interface.

Additional details of the insertion and visualization devices are disclosed in U.S. Pat. Nos. 10,624,532 and 10,398,287 and U.S. Patent Publication Nos. 2020/0113645 and 2020/0113414, the disclosure of each of which is incorporated by reference in its entirety. Additional details of controlling one or more of the tilt or pan of the distal end 224B of the camera tube are similar to those described in U.S. Patent Publication No. 2016/0143633 and U.S. Pat. No. 9,629,688, which are assigned to the assignee of the present application and the disclosure of each of which is incorporated by reference in its entirety.

### Camera Lens Cleaning

One or more of the primary camera or the secondary camera can include one or more lenses. For example, the distal end 224B of the camera tube 224 can include one or more lenses. One or more lenses of the primary or secondary camera can focus light from and/or reflected by at least the portion of the site of interest on one or more image sensors, which capture the visual representation of the site of interest. One or more lenses can include concave and/or convex lenses. In some cases, one or more lenses can be moved to adjust the zoom (such as, an optical zoom). One or more lenses can be covered with one or more protective windows (sometimes referred to as one or more windows). One or more protective windows can be transparent. When in use in or proximal to a body cavity, one or more lenses or one or more protective windows may become covered with fluids (such as, blood) or tissue, which can impair visibility, hinder the surgeon's ability to perform a surgical procedure, or endanger the patient. The one or more lenses or one or more protective windows can be cleaned by removing the camera tube 224 of the primary camera or the secondary camera from at least one of (or from both) the insertion and visualization devices. However, removing the camera can be undesirable because it can be time consuming and cumbersome. The approaches described herein facilitate cleaning the one or more lenses or one or more protective windows on demand and without the need to remove and reinsert the camera. Cleaning can include one or more of washing or wiping the one or more lenses or one or more protective windows.

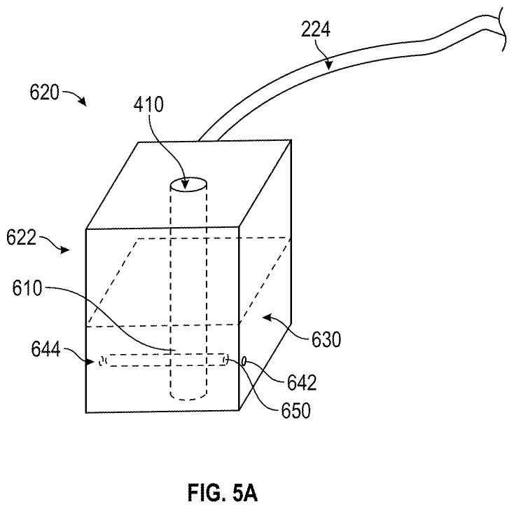

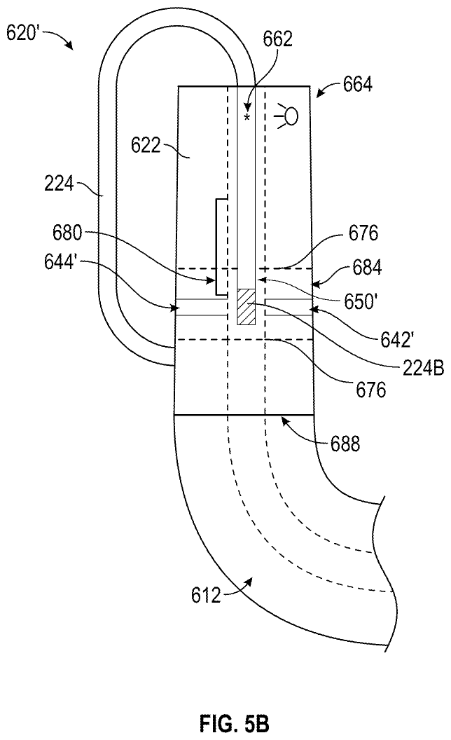

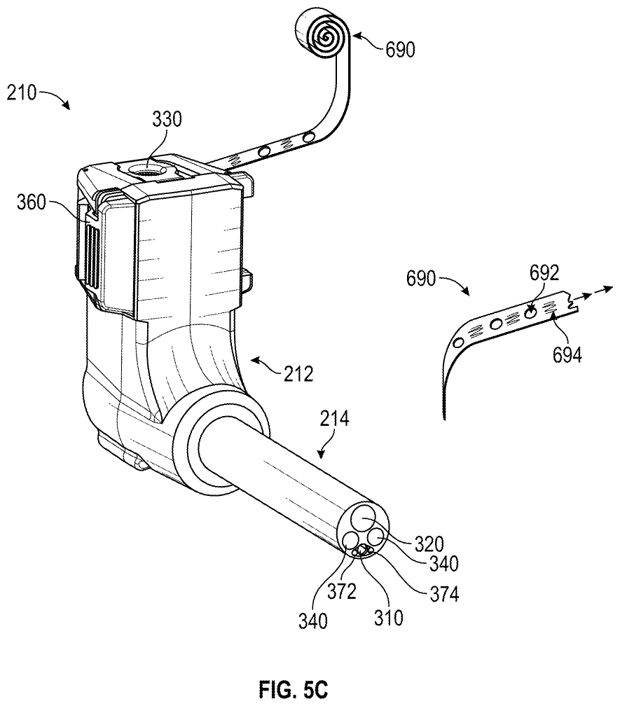

FIG. 5A illustrates a visualization device 620, which can be similar to the visualization device 220. The visualization device can include a housing 622 that, while schematically being illustrated as having a rectangular shape, can be similar to the housing 222. As descried herein, the camera tube 224 can be attached to the housing 622, and the distal end 224B of the camera tube 224 can be inserted into the housing 622 through the opening 410. The conduit 610 (which can be similar to the conduit 510) can be position in the housing 622 and connected to the opening 410. As described herein, at least the distal end 224B of the camera tube 224 can pass through the conduit 610.

One or more lenses or one or more protective windows of the camera tube 224 (which can be positioned in the distal end 224B) can be cleaned inside the conduit 610. For example, fluid (such as, saline or another washing solution) can be introduced through a fitting, port, or opening 642, which can include one or more seals. One or more lenses or one or more protective windows can be positioned and washed in a region 650, which can extend or otherwise be adjacent to the port 642 and a fitting, port, or opening 644 for removing dirty fluid or other waste. Washing can include exposing the one or more lenses or one or more protective windows to the fluid, such as, allowing the fluid to flow across the one or more lenses or one or more protective windows. Washing can include moving the distal end 224B up or down and/or rotating the distal end 224B. One or more scrubbers can be positioned in the conduit 410 for washing the one or more lenses or one or more protective windows. The region 650 can be a chamber (and may be referred to as a cleaning chamber or reservoir). The port 644 can include one or more seals. As described herein in connection with FIG. 5C, one or more wipes can be moved across the one or more lenses or one or more protective windows to one or more of clean, dry, or wipe. The one or more wipes can be replaceable. Wiping of the one or more lenses or one or more protective windows can be performed automatically (for instance, as described below) or manually.

When the camera tube 224 (such as, the distal end 224B) is positioned in a body cavity, the temperature of the camera (including one or more lenses or one or more protective windows) can be increased. Upon removal of the camera from the body cavity for cleaning, there is a risk that the one or more lenses or one or more protective windows may become covered with condensation (or fog up) due to the temperature change between the interior of the body cavity and a typically cold operating room. To prevent such undesirable occurrence, the one or more lenses or one or more protective windows can be cleaned in an environment with an increased temperature (relative to the ambient temperature of the room where the surgical procedure is being performed and where the system 100 is at least partially positioned). The increased temperature can be similar to that inside the body cavity.

In some cases, fluid can be removed using a vertical passage or channel, such as 702 in FIG. 6. In some variations, the fluid may not be removed or may be partially removed. Small amount of fluid (such as, saline) may be introduced into the patient.

Region 630 can include one or more heat generators (or devices) for warming up the one or more lenses or one or more protective windows (for instance, directly or indirectly through warming up the fluid in the chamber 650). One or more heat generators can be external to the one or more of primary camera or secondary camera. One or more heat generators can include one or more illumination sources (such as, one or more light emitting diodes (LEDs), one or more halide light source, etc.) for the one or more of a primary camera or secondary camera. It can be advantageous to direct or redirect heat produced by the one or more illumination sources for preventing one or more lenses or one or more protective windows from fogging up. In some cases, one or more heat generators can include electronic components, such as an integrated circuit component (for instance, a processor), capacitor, inductor, or the like. In some implementations, one or more dedicated heat generators can be used (such as, one or more heating elements). One or more heatsinks for transferring heat to warm up the one or more lenses or one or more protective windows can be positioned in the region 630. The camera tube 224 may not include illumination sources or other types of heat generators.

Utilizing one or more external heat generators can be advantageous for promoting patient safety. Positioning one or more heat generators within a primary or secondary camera can be risky since the one or more heat generators may malfunction inside the body cavity, which can cause discomfort or injury to the patient. With one or more external heat generators, even if a failure were to occur upon insertion of the primary or secondary camera into the body cavity, patient safety would not be comprised since there no active heating would be provided to the primary or secondary camera inserted into the body cavity.

In some cases, one or more heat generators may not be positioned in the housing 222 (or in the housing 212). For instance, a separate adapter, port, or the like with one or more heat generators can be provided to warm up the one or more lenses or one or more protective windows. A separate adapter can be connected to one or more of the housings 222 or 212 for warming up the one or more lenses or one or more protective windows.

FIG. 5B illustrates a visualization device 620′, which can be similar to the visualization device 620, connected to an insertion device (which can be similar to the insertion device 210) having a housing 612. The visualization device 620′ can have a housing 622′ (which can be similar to the housing 622). As descried herein, the camera tube 224 can be attached to the housing 622′, and the distal end 224B of the camera tube 224 can be inserted through a top opening (such as, the opening 410). At least the distal end 224B of the camera tube 224 can pass through a passage (such as, the conduit 610 connected to the opening 410).

The housing 622′ can include a cleaning chamber 650′ (sometimes referred to as a washing chamber), which can be similar to the cleaning chamber 650. The cleaning chamber 650′ can be sealed with seals 676, which are illustrated as dashed lines. The seals can be radial seals. Ports 642′ and 644′ can communicate with the chamber for the introduction and removal of fluid. A channel connected to the cleaning chamber 650′ can be formed between ports 642′ and 644′. Ports 642′ and 644′ can be similar to the ports 642 and 644. The cleaning chamber 650′ can be positioned proximate to an illumination circuit board 680, which can include one or more illumination sources and/or one or more electronic components. As described herein, heat generated by the illumination circuit board 680 can maintain the fluid in the chamber at an elevated temperature in order to prevent fogging up of the one or more lenses or one or more protective windows. One or more heat sinks or thermal transfer components (or medium) can be positioned between the illumination circuit board 680 and the cleaning chamber 650′ to facilitate the transfer of heat to the cleaning chamber 650′.

In some cases, the camera can be cleaned automatically. The camera tube 224 can include one or more fiducial points or markers 662, which can be metal objects (such as, pads), indents or other surface features, patterns or images (such as, an asterisk as shown in FIG. 5B) printed or positioned on the surface, or the like. One or more sensors 664 can be positioned inside the housing 622′ proximate to the conduit 610. The one or more sensors 664 can be optical sensors, magnetic sensors, or the like and can be configured to detect the one or more fiducial markers 662. In use, the distal end 224B of the camera tube can be positioned at a first position, which can be inside a body cavity (or another location, such as proximal to the body cavity). In the first position, the distal end 224 can be extended away from the insertion device. The camera tube 224 can be automatically retracted from the body cavity (or from another location outside the insertion device) responsive to receiving a user input, such as a press of a button (which can be positioned on the drive unit 106).

The one or more fiducial markers 662 can be positioned on the camera tube 224 at one or more locations selected such when the one or more fiducial markers 662 are positioned proximal to the one or more sensors 664, the camera (including one or more lenses or one or more protective windows) is positioned in the cleaning chamber 650′. For example, the distance between the one or more fiducial markers 662 and the tip of the camera tube 224 can be equal to (or substantially equal to) the distance between the one or more sensors 664 and the middle of the cleaning chamber 650′ (or another location in the cleaning chamber 650′). In some cases, the one or more fiducial markers 662 can be positioned in the distal end 224B of the camera tube 224, and the one or more sensors 664 can be positioned in or proximal to the cleaning chamber 650′.

The camera tube 224 can continue to be retracted until the one or more fiducial markers 662 are positioned proximal to the one or more sensors 664. Responsive to the one or more fiducial markers 662 being detected by the one or more sensors 664, retraction of the camera tube 224 can be halted. At this time, one or more camera lenses or one or more protective windows would be positioned within the cleaning chamber 650′. The chamber 650′ can be pre-filled with fluid. The one or more lenses or one or more protective windows of the camera can be cleaned and/or wiped, as described herein.

Subsequently, the camera tube 224 can be extended so that the distal end 224B is returned to the first position. For example, the number of rotations of the drive rollers 232A and 232B can be counted when the camera tube 224 is being retracted. When the camera tube is being extended, the drive rollers can be rotated for the same number of rotations to return the distal end 224B to the first position. As another example, a position sensor (such as, a position encoder) can be used to determine the position of the distal end 224B. Automatic cleaning can include wiping and/or cleaning the one or more lenses or one or more protective windows as described herein in connection with FIG. 5C. Automatic cleaning can be performed under control of an electronic processing circuitry, such as one or more processors of the electronic circuitry 118.

In some cases, the positon of the distal end 224B can be tracked or monitored. For example, a position sensor can be utilized. The position sensor can be one or more of a position encoder (such as, a rotary encoder or linear encoder), capacitive displacement sensor, eddy-current sensor, Hall effect sensor, inductive sensor, optical sensor, electrical transformer, piezo-electric sensor, potentiometer, or ultrasonic sensor. When the position of the distal end is known, one or more fiducial markers 662 and the one or more sensors 664 may be omitted. Positioning one or more camera lenses or one or more protective windows within the cleaning chamber 650′ and returning the distal end 224B to the first position can be performed using output of the position sensor. Tracking position of the distal end 224B can be used in case of a rigid camera tube 224.

The cleaning chamber 650′ is illustrated as being positioned in the visualization device. With reference to FIG. 6, which illustrates a cross-sectional view of the insertion device 210, any of the cleaning chambers described herein can be positioned in the insertion device. For example, a cleaning chamber can be positioned in the passage 322, such as in the region 702 or another region in the straight or curved part of the passage 322 or in the channel 320. In some cases, a cleaning chamber can be positioned between the visualization and insertion devices, such as in the region 688 illustrated in FIG. 5B. In some implementations, a cleaning chamber can be incorporated into a separate housing that can be, for example, positioned between the visualization and insertion devices. Such separate housing can include any of the ports, seals, conduits, or the like described and/or illustrated in connection with the camera cleaning arrangements described herein. For instance, an adapter 684 that includes the cleaning chamber 650′, ports 642′ and 644′, and seals 676 can be positioned or included in a separate housing. The adapter 684 can be disposable.

FIG. 5C illustrates a strip 690, which can be arranged on a reel or spool positioned above or below the visualization device or insertion device. The strip 690 can be made of paper or another flexible material. The strip 690 can be replaced as needed. The strip 690 can be used for cleaning and/or wiping one or more lenses or one or more protective windows of the primary camera (and/or the secondary camera). For instance, the strip 690 can include one or more cleaning portions 694 for cleaning and/or wiping the one or more lenses or one or more protective windows and one or more lumens or openings 692 for allowing the camera tube 224 of the primary camera to pass through (for instance, into the opening 330 and/or the passage 322). The openings 692 and cleaning portions 694 can alternate, as shown in FIG. 5C. One or more cleaning portions 694 (or other areas of the strip 690) can include fluid for cleaning the one or more lenses or one or more protective windows.

In use, a strip 690 can pass between the visualization device housing 222 and insertion device housing 212. The camera tube 224 can be retracted to be level (or substantially level) with the strip 690. An opening 692 can allow the camera tube 224 to be retracted without damaging the strip 690. The strip 690 can be moved (to the left or right) so that a cleaning portion 694 passes across the one or more lenses or one or more protective windows to wipe and/or clean the one or more lenses or one or more protective windows. The strip can be moved again to align the distal end 224B of the camera tube 224 with another opening 692. The camera tube 224 can be extended through such another opening 692 so that the distal end 224B is returned to the first position.

In some cases, the strip 690 can be inserted (for instance, through an opening or slot that provides access to at least a portion of the cleaning chamber) into the housing 222 of the visualization device and/or housing 212 of the insertion device. For instance, the strip 690 can be inserted into the adapter 684.

Access to any of the cleaning chambers described herein can be provided (such as, via one or more openings or ports) to clean a chamber. For instance, the chamber may be cleaned between surgeries or during a particular surgery.

Any of the cleaning chambers described herein can be positioned anywhere in the housing 212 or 222. In some cases, any of the cleaning chambers described herein can be positioned external to the housing 212 and/or 222. For instance, an adapter (such as, the adapter 684) can be attached to any of the housing 212 or 222.

### Other Variations

Those skilled in the art will appreciate that, in some embodiments, additional components and/or steps can be utilized, and disclosed components and/or steps can be combined or omitted. For example, although some embodiments are described in connection with a robotic surgery system, the disclosure is not so limited. Systems, devices, and methods described herein can be applicable to medical procedures in general, among other uses. As another example, certain components can be illustrated and/or described as being circular or cylindrical. In some implementations, the components can be additionally or alternatively include non-circular portions, such as portions having straight lines. As yet another example, any of the actuators described herein can include one or more motors, such as electrical motors.

The foregoing description details certain embodiments of the systems, devices, and methods disclosed herein. It will be appreciated, however, that no matter how detailed the foregoing appears in text, the systems, devices, and methods can be practiced in many ways. The use of particular terminology when describing certain features or aspects of the disclosure should not be taken to imply that the terminology is being redefined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures can be combined, interchanged, or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

Directional terms used herein (for example, top, bottom, side, up, down, inward, outward, etc.) are generally used with reference to the orientation or perspective shown in the figures and are not intended to be limiting. For example, positioning “above” described herein can refer to positioning below or on one of sides. Thus, features described as being “above” may be included below, on one of sides, or the like.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations).

The term “comprising” as used herein is synonymous with “including,” “containing,” or “characterized by,” and is inclusive or open-ended and does not exclude additional, unrecited elements or method steps.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function and/or achieves a desired result. For example, the terms “approximately”, “about”, “generally,” and “substantially” may refer to an amount that is within less than 10% of, within less than 5% of, within less than 1% of, within less than 0.1% of, and/or within less than 0.01% of the stated amount.

It will be further understood by those within the art that any disjunctive word and/or phrase presenting two or more alternative terms, whether in the description, claims, or drawings, can be understood to contemplate the possibilities of including one of the terms, either of the terms, or both terms. For example, the phrase “A or B” will be understood to include the possibilities of “A” or “B” or “A and B.” Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The above description discloses embodiments of systems, apparatuses, devices, methods, and materials of the present disclosure. This disclosure is susceptible to modifications in the components, parts, elements, steps, and materials, as well as alterations in the fabrication methods and equipment. Such modifications will become apparent to those skilled in the art from a consideration of this disclosure or practice of the disclosure. Consequently, it is not intended that the disclosure be limited to the specific embodiments disclosed herein, but that it cover all modifications and alternatives coming within the scope and spirit of the subject matter embodied in the following claims.

What is claimed is:

1. A visualization device for a robotic surgery apparatus, the visualization device comprising:
a housing configured to be removably attached to a mounting interface of the robotic surgery apparatus, the housing comprising first and second openings positioned on an exterior of the housing;
a camera shaft including a distal end comprising at least one camera that includes at least one lens covered by at least one window, the distal end configured to be inserted through the first opening in the housing, pass through an interior of the housing, and exit the housing through the second opening in the housing, the distal end configured to extend away from the housing toward a region of interest outside the housing and retract back toward the housing; and
a chamber positioned in the interior of the housing and configured to receive at least a portion of the distal end, the chamber further configured heat up and clean the at least one window of the at least one camera.

2. The device of claim 1, further comprising an electronic circuitry positioned in the interior of the housing and proximal to the chamber, the electronic circuitry configured generate heat that regulates temperature of the chamber to at least one of: prevent fogging of the at least one lens or the at least one window or clean the at least one window of the at least one camera.

3. The device of claim 2, wherein the electronic circuitry comprises an illumination source configured to produce illumination for the region of interest.

4. The device of claim 1, wherein the chamber is positioned in a conduit comprising the first and second openings.

5. The device of claim 4, further comprising first and second seals configured to seal off a portion of the conduit to form the chamber, wherein the chamber is configured to retain a fluid for cleaning the at least one window of the at least one camera.

6. The device of claim 5, wherein the housing further comprises first and second openings positioned at least partially on the exterior of the housing, the first opening configured to permit introduction of the fluid into the chamber and the second opening configured to permit removal of the fluid from the chamber.

7. The device of claim 1, wherein the camera shaft includes a proximal end attached to the housing.

8. The device of claim 1, wherein the camera shaft comprises a fiducial marker, and wherein the device further comprises:
a sensor configured to detect the fiducial marker; and
an electronic processing circuitry configured to:
cause the camera shaft to be retracted responsive to receiving a user input, wherein the distal end is positioned at a first position prior to retracting the camera shaft; and
cause stopping of the retraction of the camera shaft responsive to receiving a signal from the sensor indicating detection of the fiducial marker by the sensor, wherein causing the retraction of the camera shaft to be stopped causes the distal end to be positioned at a second position within the chamber.

9. The device of claim 8, wherein the electronic processing circuitry is further configured to cause extension of the camera shaft to return the distal end to the first position.

10. The device of claim 1, further comprising a position sensor configured to monitor position of the distal end and an electronic processing circuitry configured to:
cause the camera shaft to be retracted responsive to receiving a user input and further responsive to an output of the position sensor, wherein the distal end is positioned at a first position prior to retracting the camera shaft; and
cause stopping of the retraction of the camera shaft responsive to the output of the position sensor, wherein causing the retraction of the camera shaft to be stopped causes the distal end to be positioned at a second position within the chamber.

11. The device of claim 10, wherein the electronic processing circuitry is further configured to, responsive to the output of the position sensor, cause the camera shaft to be extended to position the distal end at the first position.

12. The device of claim 1, wherein the housing is configured to receive a cleaning strip that includes at least one cleaning portion configured to wipe the at least one window, and wherein the cleaning strip comprises alternating wiping portions and lumen portions, a wiping portion configured to wipe the at least one window and a lumen portion configured to allow the camera shaft to pass through and enter the first opening.

13. An insertion device for a robotic surgery apparatus, the insertion device comprising a housing including:
a first portion comprising a camera channel positioned in an interior of the first portion and extending along at least a portion of the first portion, the camera channel configured to permit insertion and removal of a primary camera shaft, the primary camera shaft including a primary camera with at least one lens covered by at least one window;
a second portion comprising a passage configured to permit the primary camera shaft to pass through, the passage aligned with the camera channel to permit at least a portion the primary camera shaft to enter the camera channel, pass through the camera channel, and exit the camera channel, the passage including an opening positioned on an exterior of the housing; and
a chamber positioned in the passage and configured to heat up and clean the at least one window of the primary camera.

14. The device of claim 13, further comprising an electronic circuitry positioned in the interior of the second portion and proximal to the chamber, the electronic circuitry configured generate heat that regulates temperature of the chamber to at least one of: prevent fogging of the at least one lens or the at least one window or clean the at least one window.

15. The device of claim 14, wherein the first portion further comprises a second camera channel configured to receive a secondary camera, and wherein the electronic circuitry comprises an illumination source configured to produce illumination for one or more of the primary camera or the secondary camera.

16. The device of claim 15, wherein the secondary camera is non-removable.

17. The device of claim 13, further comprising first and second seals configured to seal off a portion of the passage to form the chamber, wherein the chamber is configured to retain a fluid for cleaning the at least one window.

18. The device of claim 17, wherein the housing further comprises first and second openings positioned at least partially on the exterior of the first portion, the first opening configured to permit introduction of the fluid into the chamber and the second opening configured to permit removal of the fluid from the chamber.

19. The device of claim 13, wherein the primary camera comprises a fiducial marker, and wherein the device further comprises:
a sensor configured to detect the fiducial marker; and
an electronic processing circuitry configured to:
cause the primary camera shaft to be retracted responsive to receiving a user input, wherein a distal end of the primary camera shaft is positioned at a first position prior to retracting the primary camera shaft; and
cause stopping of the retraction of the primary camera shaft responsive to receiving a signal from the sensor indicating detection of the fiducial marker by the sensor, wherein causing the retraction of the primary camera shaft to be stopped causes the distal end to be positioned at a second position within the chamber.

20. The device of claim 19, wherein the electronic processing circuitry is further configured to cause extension of the primary camera shaft to return the distal end to the first position.

21. The device of claim 13, further comprising a position sensor configured to monitor position of a distal end of the primary camera shaft and an electronic processing circuitry configured to:
cause the primary camera shaft to be retracted responsive to receiving a user input and further responsive to an output of the position sensor, wherein the distal end is positioned at a first position prior to retracting the primary camera shaft; and
cause stopping of the retraction of the primary camera shaft responsive to the output of the position sensor, wherein causing the retraction of the camera shaft to be stopped causes the distal end to be positioned at a second position within the chamber.

22. The device of claim 21, wherein the electronic processing circuitry is further configured to, responsive to the output of the position sensor, cause the primary camera shaft to be extended to position the distal end at the first position.

23. The device of claim 13, wherein the housing is configured to receive a cleaning strip that includes at least one cleaning portion configured to wipe the at least one window, and wherein the cleaning strip comprises alternating wiping portions and lumen portions, a wiping portion configured to wipe the at least one window and a lumen portion configured to allow the primary camera shaft to pass through and enter the passage through the opening.
